(19) United States
(12) Patent Application Publication
(10) Pub. No.: US 2008/0305149 A1
(43) Pub. Date: Dec. 11, 2008

(54) Mucoadhesive vesicles for drug delivery

(76) Inventors: Thomas Hirt, Rebstein (CH); Zhihue Lu, Johns Creek, GA (US); Wolfgang Meier, Therwil (CH); Mansoor Amiji, Attleboro, MA (US)

Correspondence Address: LAW OFFICE OF COLLEN A. BEARD, LLC, P. O. BOX 1064, DECATUR, GA, 30031-1064, US

(21) Appl. No.: 12/157,114

(22) Filed: Jun. 6, 2008

Related U.S. Application Data
(60) Provisional application No. 60/934,034, filed on Jun. 11, 2007.

(51) Int. Cl. A61K 9/10; A61K 47/30

(52) U.S. Cl. 424/434

(57) ABSTRACT

Vesicles for delivery of active macromolecules which are formed from amphiphilic segmented copolymers having one or more mucoadhesive groups or regions and which can be used for delivery of an active agent to an area of the body having a mucous membrane, such as but not limited to the gastrointestinal tract.

### RELATED APPLICATION

The present application is related to and claims priority to U.S. Provisional Application Ser. No. 60/934,034 filed Jun. 11, 2007, the entire contents of which are incorporated herein by reference.

### BACKGROUND OF THE INVENTION

The invention is related to drug delivery and more specifically related to mucoadhesive vehicles for delivery of therapeutic and diagnostic active agents.

Mucoadhesive polymers are synthetic or natural macromolecules which are capable of physically or chemically attaching to mucosal surfaces. The concept of mucoadhesive polymers was introduced into the pharmaceutical literature more than 20 years ago and has been accepted as a promising strategy to prolong the residence time and to improve the specific location of drug delivery systems on various membranes. Since the concept of mucoadhesion was introduced, numerous attempts have been undertaken to improve the adhesive properties of polymers. These approaches include the use of linear polyethylene glycol (PEG) as an adhesion promoter for hydrogels, the neutralization of ionic polymers, mucoadhesion by a sustained hydration process, and the development of polymer adhesin (e.g., sugar-binding proteins) conjugates providing a specific binding to epithelia. All these systems are based on the formation of non-covalent bonds such as hydrogen bonds, van der Wall's forces, and ionic interactions. Accordingly, they provide only weak mucoadhesion, in many cases insufficient to guarantee the localization of a drug delivery system at a given target site.

A presumptive new generation of mucoadhesive polymers is thiolated polymers—designated thiomers. In contrast to the mucoadhesive polymers discussed above these polymers are capable of forming covalent bonds. The bridging structure most commonly encountered in biological systems—the disulfide bond—is used for the covalent adhesion of polymers to the mucous gel layer of the mucosa. Based on thiol/disulfide exchange reactions and/or a simple oxidation process, disulfide bonds are formed between such polymers and cysteine-rich subdomains of mucus glycoproteins. Hence, thiomers mimic the natural mechanism of secreted muco glycoproteins, which are also covalently anchored in the mucus layer by the formation of disulfide bonds.

Colloidal drug carriers, such as liposomes or nanoparticles of biodegradable polymers have received much attention for their ability to improve the absorption of poorly absorbable drugs, including peptide drugs. It has been reported that the mucoadhesive properties of these particulate systems can prolong their retention in the gastrointestinal tract, thus further improving drug absorption. Takeuchi et al. have demonstrated a novel mucoadhesive liposomal system prepared by coating the surface of submicron-sized liposomes with a mucoadhesive polymer, chitosan or carbopol. With both polymer coatings the enhanced and prolonged pharmacological effect of insulin was confirmed. It was also shown that the submicron sized liposomes performed much better than liposomes that were a few micrometers large. Takeuchi et al., Adv. Drug Delivery Reviews, 57 (2005): 1583-1594.

Another review describes that the presence of hydroxyl, carboxyl, or amine groups encourages adhesion to mucosal membranes. Therefore polyacrylic acid or derivatives such as Carbophil, Carbomer, and Carbopol 943 as well as chitosan, cellulose derivatives, or lecithin can show improved mucoadhesion. Smart, Adv. Drug Delivery Reviews 57 (2005): 1556-1568.

The present invention is a new approach for making mucoadhesive micro and nano size particles. The invention is hollow polymeric vesicles having mucoadhesive groups or regions. In a preferred embodiment, the vesicles are formed from an ABA, ABC, or AB amphiphilic segmented copolymer and mucoadhesion is provided by modifying the end groups of the hydrophilic A segments with hydroxyl, thiol, amine, or carboxyl groups. In contrast to liposomes these vesicles are more stable, the outer polymer shell can be chosen according to the specific needs, and the manufacturing is reproducible, since it only uses synthetic components. The vesicles can be loaded with a wide variety of active agents.

### SUMMARY OF THE INVENTION

The present invention is a drug delivery vehicle that is mucoadhesive. The vehicle comprises vesicles formed from amphiphilic segmented copolymers having one or more mucoadhesive groups or regions. The vesicles can be loaded with an active agent, or the active agent can be otherwise carried by the vesicles. The drug delivery vehicle can be used for delivery of an active agent to an area of the body having a mucous membrane, such as but not limited to the gastrointestinal tract. For example, the drug delivery vehicle can be designed for use in oral, buccal, nasal, rectal and vaginal routes for both systemic and local effects. The vesicles are made of an amphiphilic copolymer.

Various types of amphiphilic copolymers can be used. In one embodiment, the copolymer is an ABA-type copolymer, where A is hydrophilic and mucoadhesive and B is hydrophobic. A vesicle having hydrophilic inner and outer layers, mucoadhesive inner and outer layer, and a middle hydrophobic layer will be formed. The vesicle can alternatively be formed from an ABC copolymer, where both A and C are hydrophilic and A is additionally mucoadhesive, and forms the outer layer. AB copolymers can also be used, again where A is mucoadhesive and forms the outer layer. B is hydrophobic.

“Hollow particle” and “vesicle” are synonymous and refer to a particle having a hollow core or a core filled with a material to be delivered or released. Vesicles may have a spherical or other shape. They may have a unilamellar or multilamellar membrane

The terms “nanospheres” and “nanocapsules” are used synonymously herein and refer to vesicles that are stabilized through crosslinking. While the nanocapsules are generally in the nanometer size range, they can be as large as about 20 microns. Thus, the term is not limited to capsules in the nanometer size range. The capsules can be spherical in shape or can have any other shape. The terms “microspheres” and “microcapsules” may be used to refer to vesicles or capsules having a size up to about 1000 microns.

The term “polymerization” as used herein refers to end to end attachment of the amphiphilic copolymers.

The term “crosslinking” as used herein refers to interpolymer linking of all types, including end to end attachment (“polymerization”) as well as covalent or ionic bonding of any portion of a copolymer to another copolymer. Crosslinking can be through end groups or internal groups and can be via covalent, ionic, or other types of bonds.

The term “encapsulation” means incorporation of a mucoadhesive agent by any means, whether in the interior or membrane of a vesicle or nanocapsule.

The term “mucoadhesive” means a material that will adhere to mucus and thus prolong the residence of the formulation.

Multilamellar vesicles are vesicles with several concentric shells built from the segmented copolymers. These vesicles normally have a size of a few microns.

### DETAILED DESCRIPTION OF THE INVENTION

The invention is drug delivery vehicles comprising vesicles formed from amphiphilic segmented copolymers, where the outer surface of the vesicle is mucoadhesive.

Amphiphilic Copolymers and Vesicles

The formation of vesicles from amphiphilic copolymers is taught in U.S. Pat. No. 6,916,488 to Meier et al., which is useful as a guide for the invention taught herein. The formation of vesicles from the amphiphilic copolymers is a result of the amphiphilic nature of the copolymers. Aggregation of the copolymers occurs via non-covalent interactions and therefore is reversible. The vesicles can be crosslinked to provide additional stability. It should be understood that the copolymers can be polymerized via end groups, crosslinked via internal crosslinkable groups, or a combination of end group and internal group polymerization/crosslinking can be used. If the vesicles are crosslinked, the resulting nanocapsules are more stable, shape-persistent, and may preserve their hollow morphology even after they are removed from an aqueous solution.

In a preferred embodiment of the invention, segmented amphiphilic copolymers are used to form vesicles. The copolymers have the structure ABA, ABC, or AB where A and C are hydrophilic polymers and B is a hydrophobic polymer. In addition, A contains at least one mucoadhesive group or region. Under appropriate conditions, the copolymers will form vesicles having an outer hydrophilic and mucoachesive surface.

While this preferred embodiment is primarily discussed herein, the invention is not limited to this embodiment. Any segmented copolymer can be used so long as it forms a vesicle having a mucoadhesive outer surface.

The stability and integrity of a particular vesicle (and the length of time it takes to degrade) depends in a large part on the strength of the interactions between the copolymers. The strength also depends upon the stability of the junction between the hydrophilic and hydrophobic segments, and the juncture between the hydrophilic or hydrophobic segment and the polymerizing unit, if one is used. The stability further depends upon the strength of the polymerization or crosslinking, if such is used. The stability of the vesicles can be decreased by the introduction of weak links, such as biodegradable links or ionic crosslinks, between the hydrophilic and hydrophobic segments, within the hydrophilic or hydrophobic segment, or between the hydrophilic or hydrophobic segment and the polymerizing unit.

Crosslinking can be achieved using many standard techniques, including photopolymerization, for example, of acrylate groups in the presence of a photoinitiator, through the use of an alkylating agent, polyaddition reaction with diisocyanates, use of carbodiimides with dicarboxylic acids, or complexation with metal ions. Crosslinking can also be achieved using side groups and end groups which can be polymerized by free radical polymerization, side groups which can be polymerized by cationic polymerization, and side groups which can be polymerized by ring-opening polymerization or condensation reactions.

In a preferred embodiment, the vesicles are degradable. One way to design degradable vesicles is by having the bond between the A and B segments and/or the B and C segments degradable. These bonds could be enzymatically degradable (such as by having a disulphide linkage) or hydrolyzable under the conditions in the cell, e.g. pH 5.5 in the endosome. Examples of pH sensitive bonds include ester and phosphoramidate bonds. Another way to form wholly or partially degradable vesicles is to have at least one of A, B, and C degradable. It is particularly desirable that B is biodegradable so that it can be broken down and cleared by the body, however this is not an absolute requirement. Since A and C are water soluble and below 40,000 g/mol they will be cleared through the kidneys.

In addition to the hydrophilic and hydrophobic segments, the membranes may include additional hydrophobic and/or hydrophilic segments and/or pendant groups, as well as crosslinkers such as monomers or macromers with reactive groups, surfactants, and crosslinking initiators, especially photoinitiators.

Targeting or biological signal molecules can be attached to the vesicles. The surface of the vesicles can easily be modified with specific targeting ligands. This can be achieved, for example, by copolymerization with a small fraction of ligand-bearing comonomers, e.g. galactosyl-monomers. It is well known that such polymer-bound galactosyl-groups are recognized by the receptors at the surface of hepatocytes (Weigel, et al. J. Biol. Chem. 1979, 254, 10830). Such labeled vesicles will migrate to the target.

Multi-lamellar vesicles with a size of a few microns can also be used. The loading of a hydrophobic drug per volume unit is increased with multi-lamellar vesicles compared to regular vesicles and the larger size directs them to the Payer's patches for gastrointestinal delivery.

In addition to the following guidance for the selection of the hydrophilic and hydrophobic segments, selection of the polymers, molecular weights, and other aspects of the hydrophobic and hydrophilic segments is covered in U.S. Pat. No. 6,916,488 to Meier et al. and one skilled in the art can look there and elsewhere for guidance. Preparation of the copolymers and the vesicles is also taught in the Meier patent and one skilled in the art can use the teachings therein as a guide to make the vesicles.

The mean molecular weight of segment A is desirably in the range from about 1000 to about 40,000, preferably in the range from about 2000 to 20,000. B desirably has a molecular weight of about 2000 to 20,000, preferably between about 3000 and 12,000. C is desirably about 200 to 40,000, preferably between about 1000 and 20,000. A should be equal to or larger than C in order for A to form the outer surface of the vesicle.

The hollow particles to typically range from about 50 nm to about 10 micrometers in diameter, although sizes may range from about 20 nm up to about 100 microns.

Segment A

The amphiphilic segmented copolymer includes at least one segment A which includes at least one hydrophilic polymer and has groups or regions that are mucoadhesive. The mucoadhesive groups or regions can be ones that form weak, non-covalent bonds such as hydrogen bonds, van der Wall's forces, and ionic interactions, e.g. through functional groups such as hydroxyl, primary, secondary or tertiary amines, or carboxylates. Or the mucoadhesive groups or regions can be ones which form a stronger covalent bond, such as thiols. Preferred mucoadhesive polymers are thiolated polymers with thiol side chains or endgroups, e.g. poly(acrylic acid) or polycarbophil modified with cysteine.

The literature discloses several types of polymers that can improve mucoadhesion, including polythiolates, cysteine containing peptides, polyacrylates, polyacrylic acid, polyvinyl pyrrolidone (PVP), polyethylene glycol (PEG), polynucleotides, poly(hydroxyethyloxazoline), polynucleic acid, poly(hydroxyethylmethacylate), polyallylamine, polyaminoacids, polysaccharides, especially chitosan, carbophil, carbomer and carbopol, poly(dimethylaminalkyl methacrylates) and poly(dimethylaminalkyl acrylates) and the copolymers poly(dimethylaminalkyl methacrylates-co-trimethylaminoalkyl methacryalte) and poly(dimethylaminalkyl acrylates-co-trimethylaminoalkyl acryalte).

The hydrophilic segment preferably contains a predominant amount of hydrophilic monomers. A hydrophilic monomer is a monomer that typically gives a homopolymer that is soluble in water or can absorb at least 10% by weight of water.

Segment A can be a hydrophilic polymer that is inherently mucoadhesive. Examples include the list provided above and in particular include polyacrylic acid and chitosan. Segment A could instead be a hydrophilic polymer with one or more regions of mucoadhesiveness, such as if a mucoadhesive polymer is attached to the A segment.

Alternatively, Segment A can be a hydrophilic polymer that is modified with a mucoadhesive group. Such groups include thiol, hydroxyl, amine, and carboxyl groups or polymer adhesin groups (such as sugar-binding proteins or peptides). Examples of hydrophilic polymers that can be so modified include polyethylene glycol, poly(2-methyl-oxazoline), and poly(2-ethyloxazoline). The hydroxyl, amine, and carboxyl groups can be further reacted with thiol containing molecules like L-cysteine.

Several repeat units of the A segment can contain mucoadhesive groups (e.g. modification of polyacrylic acid as described in A. Bernkop-Schuerch et al., European Journal of Pharmaceutical Sciences 15 (2002) 387-394) or only the last repeat unit of A is modified with a mucoadhesive group. In a preferred embodiment, the end groups of the A segment are modified, which is especially advantageous since these end groups densely populate the surface of the vesicles formed from the amphiphilic copolymers.

End group or pendant group modification can be performed after the ABA, ABC, or AB copolymer is made, or on the A segment prior to making the copolymer.

Segment B

The amphiphilic segmented copolymer includes at least one segment B that includes a hydrophobic polymer. U.S. Pat. No. 6,916,488 to Meier et al. teaches a number of hydrophobic polymers that can be used. Examples of hydrophobic polymers that can be used, include, but are not limited to, polysiloxane such as polydimethylsiloxane and polydiphenylsiloxane, perfluoropolyether, polystyrene, polyoxypropylene, polyvinylacetate, polyoxybutylene, polyisoprene, polybutadiene, polyvinylchloride, polyalkylacrylate (PAA), polyalkylmethacrylate, polyacrylonitrile, polypropylene, PTHF, polymethacrylates, polyacrylates, polysulfones, polyvinylethers, fluoropolymers, and poly(propylene oxide), and copolymers thereof.

The hydrophobic segment preferably contains a predominant amount of hydrophobic monomers. A hydrophobic monomer is a monomer that typically gives a homopolymer that is insoluble in water and can absorb less than 10% by weight of water.

Segment C

In addition, the amphiphilic segmented copolymer may include a segment C that includes a hydrophilic polymer. U.S. Pat. No. 6,916,488 to Meier et al. teaches a number of hydrophobic polymers that can be used, such as, but not limited to, polyoxazoline, polyethylene glycol, polyethylene oxide, polyvinyl alcohol, polyvinylpyrrolidone, polyacrylamide, poly(meth)acrylic acid, polyethylene oxide-co-polypropyleneoxide block copolymers, poly (vinylether), poly(N,N-dimethylacrylamide), polyacrylic acid, polyacyl alkylene imine, polyhydroxyalkylacrylates such as hydroxyethyl methacrylate (HEMA), hydroxyethyl acrylate, and hydroxypropyl acrylate, polyols, and copolymeric mixtures of two or more of the above mentioned polymers, natural polymers such as polysaccharides and polypeptides, and copolymers thereof, and polyionic molecules such as polyallylammonium, polyethyleneimine, polyvinylbenzyltrimethylammonium, polyaniline, sulfonated polyaniline, polypyrrole, and polypyridinium, polythiophene-acetic acids, polystyrenesulfonic acids, zwitterionic molecules, and salts and copolymers thereof.

Making Vesicles

In general, vesicles can be made by a number of means known to those skilled in the art. Self assembly techniques are preferred. In one embodiment, the amphiphilic copolymer is dissolved in a solvent such as ethanol at a concentration of from about 5% to 30%. The polymer solution is then added to an aqueous solution with stirring. This procedure generally leads to a dispersion of segmented copolymer vesicles of a rather broad size distribution. The size distribution can be controlled by methods known to those skilled in the art of preparing vesicles. In addition, the size distribution can be selected by passing the polydisperse vesicles through one or more filters having a defined pore size. The resulting vesicle dimensions are directly determined by the pore diameter of the filter membrane.

Active Agents

Examples of active agents that can be used with the mucoadhesive vesicles are labile molecules, proteins, and molecules with low bioavailability. In particular, the invention is targeted to oral, nasal and buccal delivery of drugs such as Fosamax, Insulin, peptides, DNA, RNA, and oligonucleotides.

The vehicles are suitable for delivery of nearly every type of active agent including therapeutic, diagnostic, or prophylactic agents as well as many compounds having cosmetic and industrial use, including dyes and pigments, fragrances, cosmetics, and inks. Both hydrophilic and hydrophobic drugs, and large and small molecular weight compounds, can be delivered. Drugs can be proteins or peptides, polysaccharides, lipids, nucleic acid molecules, or synthetic organic molecules. Examples of hydrophilic molecules include most proteins and polysaccharides and oligonucleotides. Examples of hydrophobic compounds include some chemotherapeutic agents such as cyclosporine and paclitaxel. Agents that can be delivered include nucleic acids therapeutics such as oligonucleotides, small interference RNA (siRNA), and genes, pain medications, anti-infectives, hormones, chemotherapeutics, antibiotics, antivirals, antifungals, vasoactive compounds, immunomodulatory compounds, vaccines, local anesthetics, angiogenic and antiangiogenic agents, antibodies, anti-inflammatories, neurotransmitters, psychoactive drugs, drugs affecting reproductive organs, and antisense oligonucleotides. Diagnostic agents include gas, radiolabels, magnetic particles, radioopaque compounds, and other materials known to those skilled in the art.

Although described here primarily with reference to drugs, it should be understood that the vesicles can be used for delivery of a wide variety of agents, not just therapeutic or diagnostic agents. Examples include cosmetic agents, fragrances, dyes, pigments, photoactive compounds, and chemical reagents, and other materials requiring a controlled delivery system. Other examples include metal particles, biological polymers, nanoparticles, biological organelles, and cell organelles.

Large quantities of therapeutic substances can be incorporated into the central cavity of the vesicles. Active agents can be encapsulated into the polymer by different routes. In one method, the agent may be directly added to the copolymer during preparation of the copolymer. For example, the compound may be dissolved together with the polymer in ethanol. In a second method, the drug is incorporated into the copolymer after assembly and optionally covalent crosslinking. The hollow particles can be isolated from the aqueous solution and redissolved in a solvent such as ethanol. Ethanol is a good solvent for the hydrophilic and the hydrophobic parts of some polymers. Hence, the polymer shell of the hollow particles swells in ethanol and becomes permeable. Transferring the particles back into water decreases the permeability of the shell.

Vesicles that are made from non crosslinked, self assembling segmented copolymers can be loaded through methods known to those skilled in the art, such as by contacting the vesicles with a solution of the active agent until the agent has been absorbed into the vesicles, the solvent exchange method or the rehydration method.

### EXAMPLES

Different difunctional triblock amphiphilic segmented copolymers can be synthesized by terminating living cationic polymerization of 2-methyl oxazoline initiated by difunctional polydimethylsiloxane with various functional small molecules.

### Example 1

### Synthesis of HO-PMOXA-PDMS-PMOXA-OH

This is an example of a hydroxylated triblock amphiphilic segmented copolymer.

Bifunctional poly(dimethylsiloxane)

Diamino functional PDMS (Mn 1600, Shin-Etsu Silicones of America) is converted to dichloroalkyl PDMS by reacting with a 10% excess chloromethylbenzoyl chloride. Diamino PDMS (96.0 g) is dried at 40 C under vacuum for 12 h before 100 ml of dry 1,2-ethylene dichloride and 12 ml dry triethylamine are added under nitrogen. 25.0 g of p-chloromethylbenzoyl chloride in 10 ml of dry 1,2-ethylene dichloride is added dropwise into the PDMS solution at 5 C. The mixture is allowed to warm up to room temperature and stirred for 12 h. Ethanol (5 ml) is added to convert the excess chloromethylbenzoyl chloride by stirring for 6 h. The mixture is diluted with 200 ml of hexane and filtered. The solution is washed with water three times and dried by anhydrous MgSO₄. After filtration, the solvent is removed under reduced pressure and vacuum; the crude product is further purified by alcohol/ethyl acetate extraction three times. The solvents are removed under vacuum.

PMOXA-PDMS-PMOXA Triblock Copolymer with Free Hydroxyl End Groups.

Poly(2-methyloxazoline)-block-poly(dimethylsiloxane)-block-poly(2-methyloxazoline) triblock polymer is made under anhydrous condition. Dichloro functional PDMS macromer (7.0 g) is dried at 60 C overnight under vacuum. 100 ml of dry chloroform and 3.0 ml of 2-methyl oxazoline are added into the macromer flask under nitrogen. Catalyst potassium iodide (1.0 g) is dried overnight under vacuum and dissolved in 100 ml of dry acetonitrile before being transferred into the reaction flask. The living polymerization is carried out under nitrogen at 70 C for 24 h. The mixture is cooled down to room temperature and then the reaction is terminated by adding potassium hydroxide (0.48 g) in 5 ml of alcohol and stirring for 3 h. After removal of solvent under reduced pressure, the product is dissolved in 100 ml of alcohol and purified by diafiltration through regenerated cellulose membrane (Millipore, molecular weight cutoff 1K) using over 600 ml of alcohol. The solvent is removed under reduced pressure and the resulting polymer is dried under vacuum.

### Example 2

### Synthesis of H2N-PMOXA-PDMS-PMOXA-NH2

This is an example of a triblock amphiphilic segmented copolymer having amine end groups. Dichloro functional PDMS macromer (Mn 1900, 20.0 g) is dried at 60 C overnight under vacuum. 100 ml of dry chloroform and 2-methyl oxazoline (9.60 g) are added into the macromer flask under nitrogen. Catalyst potassium iodide (1.72 g) is dried overnight under vacuum and dissolved in 100 ml of dry acetonitrile before being transferred into the reaction flask. The living polymerization is carried out under nitrogen at 80 C for 24 h. The mixture is cooled down to room temperature and then the reaction is terminated by adding 5.0 ml of 7N ammonia in methanol and stirring for 3 h. After removal of solvent under reduced pressure, the product is dissolved in 200 ml of alcohol/water (1:1, v/v) and purified by diafiltration through regenerated cellulose membrane (Millipore, molecular weight cutoff 1K) using over 1000 ml of alcohol/water (1:1, v/v). The solvent is removed by under reduced pressure and the resulting polymer is dried under vacuum.

### Example 3

### Synthesis of NHR-PMOXA-PDMS-PMOXA-NHR

This is an example of a triblock amphiphilic segmented copolymer having secondary amine end groups. Dichloro functional PDMS macromer (Mn 1900, 20.0 g) is dried at 60 C overnight under vacuum. 100 ml of dry chloroform and 2-methyl oxazoline (9.60 g) are added into the macromer flask under nitrogen. Catalyst potassium iodide (1.72 g) is dried overnight under vacuum and dissolved in 100 ml of dry acetonitrile before being transferred into the reaction flask. The living polymerization is carried out under nitrogen at 80 C for 24 h. The mixture is cooled down to room temperature and then the reaction is terminated by adding 1-boc-piperazine (3.91 g) in 20 ml of alcohol and stirring for 1 h. After removal of solvent under reduced pressure, the product is dispersed in 800 ml of alcohol/water (1:7, v/v). Concentrated hydrochloric acid (37%, 5 ml) is added and the mixture stirred for 1 h, followed by adding 2.5N sodium hydroxide until pH 9. The resulting polymer is separated and purified by diafiltration through regenerated cellulose membrane (Millipore, molecular weight cutoff 1K) using over 1000 ml of alcohol/water (1:1, v/v). The solvent is removed under reduced pressure and the resulting polymer is dried vacuum.

### Example 4

### Synthesis of HS-PMOXA-PDMS-PMOXA-SH

This is an example of a triblock amphiphilic segmented copolymer having thiol end groups. Dichloro functional PDMS macromer (Mn 1900, 15.0 g) is dried at 60 C overnight under vacuum. 150 ml of dry chloroform and 2-methyl oxazoline (6.45 g) are added into the macromer flask under nitrogen. Catalyst potassium iodide (1.25 g) is dried overnight under vacuum and dissolved in 100 ml of dry acetonitrile before being transferred into the reaction flask. The living polymerization is carried out under nitrogen at 80 C for 24 h and terminated by adding sodium hydrosulfide (2.60 g) in 40 ml of alcohol and stirring for 3 h. After removal of solvent under reduced pressure, the product is dissolved in 100 ml of alcohol/water (1:1, v/v) and purified by diafiltration through regenerated cellulose membrane (Millipore, molecular weight cutoff 1K) using over 1000 ml of alcohol/water (1:1/v/v). The solvent is removed under reduced pressure and the resulting polymer is dried vacuum.

### Example 5

### Synthesis of NaOOC-PMOXA-PDMS-PMOXA-COONa

This is an example of a triblock amphiphilic segmented copolymer having carboxyl end groups. HO-PMOXA-PDMS-PMOXA-OH prepared as in Example 1 (9.0 g) is dried at 60 C under vacuum for 12 h and dissolved in 100 ml of dry chloroform. Succinic anhydride (1.26 g) was added and the solution stirred at 70 C for 24 h. After removal of solvent under reduced pressure and vacuum, the product is dispersed in 100 ml of alcohol/water (1:7, v/v) and 2.5N sodium hydroxide is added to reach pH 8. The resulting polymer is separated and purified by diafiltration through regenerated cellulose membrane (Millipore, molecular weight cutoff 1K) using over 800 ml of alcohol/water (1:1, v/v). The solvent is removed under reduced pressure and the resulting polymer is dried vacuum.

### Example 6

### Making Vesicles

The block copolymer (0.5 g) is dissolved in 0.5 ml of EtOH and then slowly dropped into 50 ml of distilled water or 2 mM PBS solution while stirring. Triblock film or powder can also be directly dispersed in distilled water or PBS water to form vesicles. Afterwards the solution is extruded through 0.45 um and 0.2 um filters. The received solution is cleaned over a Sepharose® 4B column in order to separate the vesicles from other aggregates such as micelles if necessary.

Modifications and variations of the present invention will be apparent to those skilled in the art from the forgoing detailed description. All modifications and variations are intended to be encompassed by the following claims. All publications, patents, and patent applications cited herein are hereby incorporated by reference in their entirety.

What is claimed is:

1. A drug delivery vehicle, comprising mucoadhesive vesicles made from amphiphilic segmented copolymers having an AB, ABA, or ABC structure, wherein segments A and C are each hydrophilic, segment B is hydrophobic, and at least one segment is mucoadhesive.

2. The drug delivery vehicle of claim 1 wherein segment A has at least one mucoadhesive region or endgroup.

3. The drug delivery vehicle of claim 2 wherein the mucoadhesive endgroup of segment A is a hydroxyl, thiol, amine, or carboxyl group.

4. The drug delivery vehicle of claim 3 wherein segment A is poly(2-methyl-oxazoline) or poly(2-ethyl oxazoline).

5. The drug delivery vehicle of claim 1 wherein segment A is mucoadhesive and is selected from the group consisting of polythiolates, cysteine containing peptides, polyacrylates, polyacrylic acid, polyvinyl pyrrolidone (PVP), polyethylene glycol (PEG), polynucleotides, poly(hydroxyethyloxazoline), polynucleic acid, poly(hydroxyethylmethacylate), polyallylamine, polyaminoacids, polysaccharides, especially chitosan, carbophil, carbomer and carbopol, poly(dimethylaminalkyl methacrylates), poly(dimethylaminalkyl acrylates), and the copolymers poly(dimethylaminalkyl methacrylates-co-trimethylaminoalkyl methacryalte) and poly(dimethylaminalkyl acrylates-co-trimethylaminoalkyl acryalte).

6. The drug delivery vehicle of claim 5 wherein segment A is polyacrylic acid or chitosan.

7. The drug delivery vehicle of claim 1 wherein the amphiphilic segmented copolymer is biodegradable.

8. The drug delivery vehicle of claim 1 wherein the link between segment A and segment B or segment B and segment C is biodegradable and segment B is biodegradable.

9. The drug delivery vehicle of claim 1 wherein segment B is biodegradable.

10. The drug delivery vehicle of claim 1 wherein segment A has a molecular weight between about 1000 to 40,000, segment B has a molecular weight between about 2000 to 20,000, and segment C has a molecular weight between about 200 and 40,000.

11. The drug delivery vehicle of claim 10 wherein segment A has a molecular weight between about 2000 to 20,000, segment B has a molecular weight between about 3000 to 12,000, and segment C has a molecular weight between about 1000 and 20,000.

12. The drug delivery vehicle of claim 1 wherein the vesicles range from about 20 nm up to about 100 microns in diameter.

13. The drug delivery vehicle of claim 1 formulated for oral, nasal, or buccal delivery.

14. An amphiphilic segmented copolymer for forming mucoadhesive drug delivery vesicles, having an AB, ABA, or ABC structure, wherein segments A and C are each hydrophilic, segment B is hydrophobic, and at least one segment is mucoadhesive.

15. The amphiphilic segmented copolymer of claim 14 wherein segment A has at least one mucoadhesive region or endgroup.

16. The amphiphilic segmented copolymer of claim 15 wherein the mucoadhesive endgroup of segment A is a hydroxyl, thiol, amine, or carboxyl group.

17. The amphiphilic segmented copolymer of claim 16 wherein segment A is poly(2-methyl-oxazoline) or poly(2-ethyl oxazoline).

18. The amphiphilic segmented copolymer of claim 14 wherein segment A is mucoadhesive and is selected from the group consisting of polythiolates, cysteine containing peptides, polyacrylates, polyacrylic acid, polyvinyl pyrrolidone (PVP), polyethylene glycol (PEG), polynucleotides, poly(hydroxyethyloxazoline), polynucleic acid, poly(hydroxyethylmethacylate), polyallylamine, polyaminoacids, and polysaccharides.

19. The amphiphilic segmented copolymer of claim 18 wherein segment A is polyacrylic acid, chitosan, or poly(dimethylaminoethyl methacylate).

20. The amphiphilic segmented copolymer of claim 14 wherein the amphiphilic segmented copolymer is biodegradable.

21. The amphiphilic segmented copolymer of claim 14 wherein the link between segment A and segment B or segment B and segment C is biodegradable and segment B is biodegradable.

22. The amphiphilic segmented copolymer of claim 14 wherein segment B is biodegradable.

23. The amphiphilic segmented copolymer of claim 14 wherein segment A has a molecular weight between about 1000 to 40,000, segment B has a molecular weight between about 2000 to 20,000, and segment C has a molecular weight between about 200 and 40,000.

24. The amphiphilic segmented copolymer of claim 23 wherein segment A has a molecular weight between about 2000 to 20,000, segment B has a molecular weight between about 3000 to 12,000, and segment C has a molecular weight between about 1000 and 20,000.
